(19) United States
(12) Patent Application Publication
(10) Pub. No.: US 2021/0236438 A1
(43) Pub. Date: Aug. 5, 2021

(54) COMPOUNDS AND METHODS FOR THE TREATMENT OF AUTISM SPECTRUM DISORDER AND OTHER NEUROLOGICAL OR PSYCHIATRIC DISORDERS

(71) Applicants: Tufts Medical Center, Inc., Boston, MA (US); Children's Medical Center Corporation, Boston, MA (US)

(72) Inventors: Isabelle Draper, Andover, MA (US); Alan S. Kopin, Wellesley, MA (US); Matthew S. Alexander, Boston, MA (US); Louis M. Kunkel, Boston, MA (US)

(21) Appl. No.: 15/734,869

(22) PCT Filed: Jun. 5, 2019
(86) PCT No.: PCT/US2019/035659

§ 371 Date: Dec. 3, 2020

Related U.S. Application Data
(60) Provisional application No. 62/681,070, filed on Jun. 5, 2018.

(51) Int. Cl. A61K 31/122; C12Q 1/6883

(52) U.S. Cl. CPC A61K 31/122; C12Q 1/6883; C12Q 2600/156; C12Q 2600/158; C12Q 2600/106

(57) ABSTRACT

Disclosed are methods of treating autism spectrum disorder (ASD) by administering a therapeutically effective amount of an isoprenoid antibiotic. Also disclosed are methods of modulating (either upregulation or downregulation) hnRNP L and hnRNP L targets by administering a therapeutically effective amount of an isoprenoid antibiotic. Methods of screening compounds for use in treating autism spectrum disorder (ASD) are also described.

### FIELD OF THE INVENTION

The present invention relates to treatment of neurological disorders.

### BACKGROUND

There is a high incidence of autism spectrum disorder (ASD) in the general population (˜1 in 68 children). Very few therapeutics have effects on the primary symptoms of autism spectrum disorder (and correlated neurological conditions e.g. attention-deficit/hyperactivity disorder (ADHD), epilepsy, mental retardation, intellectual disability), including those typically used for neuropsychiatric disorders. There exists a need for safe compounds for the treatment of autism spectrum disorder.

### SUMMARY OF THE INVENTION

The invention provides a compounds, compositions, and methods for the treatment, e.g., reduction of symptoms, of autism spectrum disorder (ASD) as well as other neurological and/or psychological disorders or conditions. Exemplary neurological and psychiatric disorders include, but are not limited to, autism, autism spectrum disorder, mental retardation, learning disability, intellectual disability, attention-deficit/hyperactivity disorder (ADHD), dyslexia, epilepsy, bipolar disorder, and schizophrenia.

A method for reducing a symptom of Autism Spectrum Disorder (ASD) is carried out by administering to a subject a composition that upregulates or downregulates heterogeneous nuclear ribonucleoprotein L (hnRNP L) or a downstream target of hnRNP L. The subject to be treated is identified by behavioral indications and/or aberrant expression of hnRNP L and/or genetic abnormalities or aberrant expression/splicing in a downstream target of hnRNP L, e.g., RbFox1 or Bin1. Compounds that upregulate hnRNP L are also useful to treat, e.g., reduce the symptoms, of intellectual disability as well as neurodegenerative disease such as Alzheimer's Disease (AD) or dementia. Subjects to be treated for AD are identified by clinical diagnosis and/or detection of a neurochemical biomarker in the cerebrospinal fluid (e.g. β-amyloid peptides, total tau and phospho-tau) and/or identification of a genetic biomarker (e.g., PSEN1, PSEN2, APOE ε4, BIN1 alleles) (Humpel, Trends Biotechnol, 2011 January; 29(1): 26-32; Huynh and Mohan, March 20; 8:102. 2017) and/or aberrant expression of hnRNP L. Such compounds are also useful for maladies such as dementia and/or brain injury/concussion, e.g., disorders involving a role of tau (MAPT), e.g., in frontotemporal dementia.

RNA is useful in detection of defects in splicing (spliceopathy), e.g., abnormal regulation of alternative splicing. The invention includes methods of detection of such defects as well as therapeutic methods for treating the defects. Protein and nucleic acid sequences useful in such therapeutic methods include: mRNA: Homo sapiens heterogeneous nuclear ribonucleoprotein L (HNRNPL), transcript variant 1, mRNA, 2,129 bp linear mRNA Accession: NM_001533.2 GI: 52632382; Homo sapiens heterogeneous nuclear ribonucleoprotein L (HNRNPL), transcript variant 2, mRNA 1,895 bp linear mRNA, Accession: NM_001005335.1 GI: 52632384. Useful protein/polypeptide sequences include: heterogeneous nuclear ribonucleoprotein L isoform a [Homo sapiens], 589 aa protein, Accession: NP_001524.2 GI: 52632383; heterogeneous nuclear ribonucleoprotein L isoform b [Homo sapiens], 456 aa protein, Accession: NP_001005335.1 GI: 52632385.

The method optionally includes a step wherein the downstream target comprises BIN1, e.g., BIN1mRNA, Homo sapiens bridging integrator 1 (BIN1), transcript variant 1, mRNA, 2,686 bp linear mRNA, Accession: NM_139343.2 GI: 346716175; Ensembl:ENSG00000136717. The sequence of BIN1 Protein, myc box-dependent-interacting protein 1 isoform 1 [Homo sapiens], 593 aa protein, is available at Accession: NP_647593.1 GI: 21536400. Protein isoforms 2-16 are also available in the art. In addition to BIN1, other targets include TTN and DMD (see table below).

In some embodiments, the downstream targets include transcripts that are also targets of FOX1 (RBFOX1/A2BP1, Accession No. NP_665898.1). We have shown that hnRNP L and FOX1 co-immunoprecipitate (i.e., these splicing factors likely function together; FIG. 3). Aberrant splicing of FOX1 targets (due to a mutation in FOX1, leading in turn to ASD) is rescued by hnRNP L if the targets are downstream of both splicing factors.

Compositions such as small molecule drugs, e.g., ascochlorin, are also useful to treat spliceopathies. In preferred embodiments, the composition increases the level of hnRNP L in a neuronal cell of the subject. For example, the composition comprises an isoprenoid antibiotic such as ascochlorin or derivatives thereof. Delivery routes include intrathecal, intravenous (IV), sub-cutaneous, oral, skin patch, nasal aerosol. Preferably, route of administration comprises least intrusive modes of delivery such as oral administration.

A preferred composition includes ascochlorin or a derivative thereof. Exemplary derivative compounds include an ascochlorin glycoside Vertihemipterin A, a aglycone thereof, 4′,5′-dihydro-4′-hydroxyascochlorin, 8′-hydroxyascochlorin; LL-Z1272delta, 8′,9′-dehydroascochlorin, ascofuranone, ascofuranol, AS-6, Cylindrol A5, 4-O-methylascochhlorin (MAC), or colletochlorin. Particularly preferred are compounds characterized as having minimal or absence of toxicity. For example, MAC has been tested in clinical trials (U.S. Pat. No. 3,995,061, 1976) and was well tolerated. The ascochlorin derivatives 4-O-methyl-ascochlorin (MAC), and 4-O-ethyl-ascochlorin display low toxicity as assessed by high LD50 after ip or oral administration (Hosokawa T et al., U.S. Pat. No. 3,995,061, 1976). Suitable compound include 4-O-methylascochlorin (MAC), 4-O-ethylascochlorin, and other derivatives/analogs, including AS-6, ascofuranone (AF) and AF-like analogs/ubiquinol mimics isolated via novel routes of synthesis using structure activity relationships (SAR) (e.g., AF-like analogues 18 and 19, as described in West et al., Eur J Med Chem. 2017 Dec. 1; 141:676-689), ascochlorin glycoside Vertihemipterin A, a aglycone thereof, 4′,5′-dihydro-4′-hydroxyascochlorin, 8′-hydroxyascochlorin; LL-Z1272delta, 8′,9′-dehydroascochlorin.

In addition to therapeutic method, screening methods are encompassed by the invention. For example, a method of identifying a compound for treatment of ASD (and other disorders/diseases described above), include steps of contacting a neuronal cell with a candidate compound and detecting an increase in hnRNP L expression or activity or an increase in expression or activity of a downstream target of hnRNP L, wherein the increase indicates that the compound decreases a symptom or severity of ASD as well as other disorders/diseases as listed above). Suitable exemplary cell lines include mammalian/human (normal or diseased) iPS cells-derived neuroprogenitor, neuron, or glia including oligodendrocytes, astrocytes (e.g., GIBCO® Human Neural Stem Cells (hNSCs, embryonic H9-derived), rat fetal neural stem cells, rat glial precusor cells (rGPC); rat adrenal gland phaeochromocytoma PC-12 cell line); primary neurons/glia cultures; immortalized neuronal cell lines (e.g., neuroblastoma cell lines: human SH-SY5Y, human SK-N-AS, hybrid rat/mouse F11; or mouse hippocampal neuronal HT-22 cell line).

Diagnostic methods are also encompassed. For example, a method for identifying a subject suffering from or at risk of developing ASD (other disorders/diseases described above), comprise the steps of detecting a mutation/defect in the hnRNP L gene/mRNA/protein in a tissue/cells of the subject. Suitable patient-derived samples to be evaluated include those obtained in a minimally or non-invasive manner, e.g., bodily fluids as well as cell or tissue samples, as described below.

The nature of DNA/RNA mutation includes but is not limited to: deletion, insertion, point mutation, missense mutation, sense mutation, single nucleotide polymorphism, splice site mutation, cryptic splice site recruitment, mutation in RNA binding protein (RBP) binding sequence. The nature of the protein defect includes but is not limited to: truncation, elongation, amino acid change, aberrant post-translation modification. Assessment includes using minimally invasive procedures, e.g., using DNA from hair, skin cells, saliva, blood, iPS cells derived differentiated cells (e.g., neurons).

A method for identifying a subject suffering from or at risk of developing ASD (or other disorders/diseases listed above), comprising detecting an alteration (or a change) in hnRNP L level (RNA, protein or activity, versus normal) or an alteration (or a change) in the normal hnRNP L mRNA variant/protein isoform (e.g., expression of the fetal isoform versus adult isoform) in a tissue of the subject compared to a normal control hnRNP L level, wherein a decrease or increase of at least 10% compared to a normal control level indicates that the subject comprises or is at risk of developing ASD. For example, an increase may reflect a compensatory mechanism linked to the pathogenesis. In some cases, both increased level as well as decreased level of splicing factors (other than hnRNP L) have been linked to pathogenesis, e.g., decrease in RBFOX1 linked to heart failure (Gao et al., J Clin Invest. 2016 January; 126(1):195-206).

Assessment of differential hnRNP L expression, examples include but are not limited to mRNA levels, e.g., quantitative RT-PCR analysis using hnRNP L-specific primers (e.g., Origene HNRNPL Human qPCR Primer Pair (NM_001533) cat #HP228107); TwistDx™ isothermal nucleic acid amplification technology that enables combination of primers and detection of multiple hnRNP L variants. For determination of protein levels, assays, e.g., Western blot analysis, using a commercially available anti-human hnRNP L antibody (monoclonal, e.g., clone 4D11, or polyclonal) are useful. Methods are used to assess activity levels, e.g., hnRNP L variants with higher or lower activity.

A method for identifying in a subject suffering from or at risk of developing ASD (or other disorders/diseases) also include evaluation of efficacy of therapeutic treatment, comprising partial of at least 10%, or complete, restoration of normal hnRNP L level (RNA, protein or activity) or hnRNP L mRNA variant/protein isoform expression pattern, in a tissue of a subject where normal is defined as control values found in corresponding normal human tissue.

The method of treatment describes herein may further comprise administering to a subject a composition that upregulates heterogeneous nuclear ribonucleoprotein L (hnRNP L) or restores normal splicing in a downstream target of hnRNP L that is misspliced as a result of a genetic mutation in the target gene, or subsequently to the neurological pathogenesis related to ASD or other disorders/diseases.

The invention includes methods of diagnosis that include the steps of contacting a tissue or bodily fluid sample from a subject with an hnRNP L binding agent and a detectable label to form a complex and measuring the amount of the complex. Suitable reagents include but are not limited to a Tagged/flagged/radiolabeled anti-hnRNP L antibody, a Tagged/flagged/radiolabeled short nucleotide sequence that binds hnRNP L (e.g., CACA repeats, or derived from a known hnRNP L RNA target), Tagged/flagged protein partner (e.g., RBFOX1), or short peptide derived-thereof, and Tagged/flagged nucleotide sequence derived from hnRNP L RNA (based on documented autoregulation). Tagged or flagged reagents are those that are labelled with a radioactive compound visually detectable reagent such as a fluorescent compound (whether the reagent is directly labeled, or by using a secondary conjugated (e.g., Alexa, Cy3, Cy5) antibody directed against the reagent), or that can be detected using a colorimetric assay (e.g., ELISA). Such detection methods are also useful in a method of monitoring disease severity or response to treatment, comprising measuring an amount of hnRNP L level (mRNA, protein, activity) in a tissue of a subject following administration of a medicament, wherein an increase over time indicates that the disease severity is decreasing in response to treatment.

The invention also includes methods of diagnosis that include the steps of contacting a tissue or bodily fluid sample from a subject with altered splicing, or expression level, or variant/isoform expression profile, of a target of hnRNP L (e.g., BIN1, DMD, TTN) to determine a subject suitable for treatment or assess progress/efficacy of treatment. The invention also includes methods of assessing efficacy of a therapeutic agent on the above hnRNP L or target of hnRNP L, that include the steps of contacting a tissue or bodily fluid sample from a subject, and showing restoration of the normal expression level or splicing or variant/isoform expression profile.

Each embodiment disclosed herein is contemplated as being applicable to each of the other disclosed embodiments.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

All references, e.g., journal articles, protein or nucleic acid sequence accession numbers, cited U.S. patents, U.S. patent application publications and PCT patent applications designating the U.S., are hereby incorporated by reference in their entirety.

### DESCRIPTION OF THE DRAWINGS

FIG. 1 is a diagram showing that hnRNP L plays a key role in brain pathophysiology. The 10 nucleotide consensus hnRNP L target sequence, and exemplary 10 hnRNP L target sequences (with high-score motifs in red or in bold, low-score motifs underlined), and 2 control sequences (with asterisks; #20 or SEQ ID NO: 11 and #15 or SEQ ID NO: 13) with their K_{D} values are given. (See Hui et al. EMBO J. 24(11): 1988-98, 2005.) In some embodiments, the hnRNP L binding site, described herein, comprises a consensus amino acid sequence shown in the above sequence logo plot. For example, such high-affinity hnRNP L binding sites may comprise between two and four motifs, mostly of the high-score type (ACAC, CACA): SEQ ID NO: 1, SEQ ID NO: 2, SEQ ID NO: 3, and SEQ ID NO: 4. For example, such intermediate-affinity hnRNP L binding sites may comprise only one or two high-score motifs each (ACAC, CACA): SEQ ID NO: 5, SEQ ID NO: 6, and SEQ ID NO: 7. For example, such low-affinity hnRNP L binding sites may comprise only a single, low-score motif (TACA, CACC): SEQ ID NO: 8, SEQ ID NO: 9, and SEQ ID NO: 10.

| Hui # | Sequence | SEQ ID NO: |
|---|---|---|
| 51 | ATACATGACACACACACGCA | 1 |
| 55 | CACACACACTACACCACAC | 2 |
| 1 | CACACCACAACGGCACCACA | 3 |
| 63 | TGCATCTACGTACACACACA | 4 |
| 33 | AATCACATACGCTTTACACA | 5 |
| 105 | CCGGACAAACACACGAACAC | 6 |
| 26 | TGCAAATGAAATGACTACAC | 7 |
| 72 | TTCACCTTTACCGGCAGCCT | 8 |
| 49 | TGCAAACCTAAATGAGGTACA | 9 |
| 23 | AAACCACCCCAAACAACAAC | 10 |
| 20* | TTTACGACTCTGCACGTTAA | 11 |
| 6 | ATGCAGCCCTCGTCATTGGC | 12 |
| 15* | TTGTTCCGATATAGCGCGAC | 13 |

FIG. 2 is a series of photographs showing that ascochlorin treatment increases the level of hnRNP L in rat embryonic cortical neurons.

FIG. 3 is a photograph showing that HNRNP L interacts with RBFOX1.

FIG. 4 is a table showing the absence of significant toxicity with administration of ascochlorin/derivatives in multiple disease models.

FIG. 5 is a table of ascochlorin and derivatives thereof and corresponding chemical structures.

FIG. 6 is a table showing that the ascochlorin derivatives 4-O-methyl-ascochlorin (MAC), and 4-O-ethyl-ascochlorin display low toxicity as assessed by high LD50 after ip or oral administration.

FIG. 7 is a diagram showing compounds 18 and 19 from West et al. Eur. J. Med. Chem. 2017.

FIG. 8 is a diagram showing ascochlorin derivatives/analogs and corresponding chemical structures.

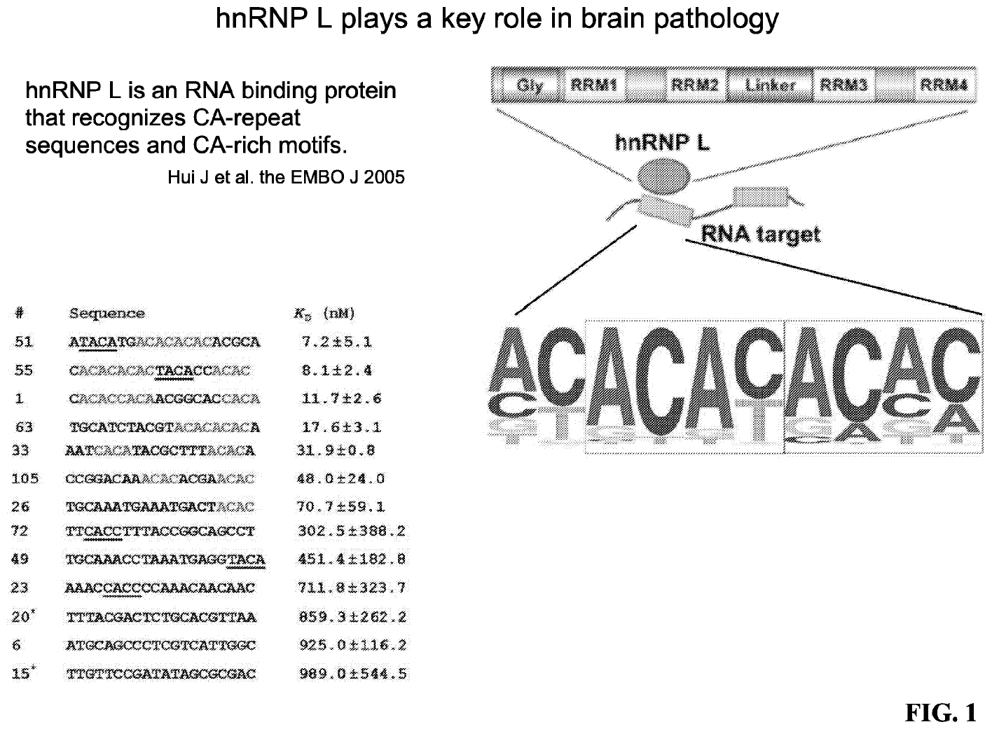

### DETAILED DESCRIPTION

Described herein is a conserved pathway downstream of the splicing factor hnRNP L, which comprises 46 genes that are conserved from invertebrates to mammals including humans. A bioinformatic analysis of a compendium of known human alternative splicing events was carried out to identify sequences that include the hnRNP L binding motif and are expressed in skeletal muscle and/or the heart. This analysis revealed 46 conserved putative targets of hnRNP L in muscle and the heart. (See U.S. Pat. No. 9,662,314, 2017.)

Table of 46 genes

| Human | | Ensembl | Accession |
|---|---|---|---|
| Gene | Description | Gene ID | Number * |
| AB11 | ABL interactor | Ensembl:ENSG | NP_005461.2 |
| | 1/spectrin SH3 | 00000097007 | |
| ACSS2 | Acetyl | Ensembl:ENSG | NP_001070020.2 |
| | Coenzyme A | 00000131069 | |
| | synthetase | | |
| | ArfGAP GTPase | | |
| AGAP3 | ankyrin repeat and | Ensembl:ENSG | NP_114152.3 |
| | PH domain | 00000133612 | |
| | containing | | |
| AGXT2L2 | Alanine glyoxylate | Ensembl:ENSG | NP_699204.1 |
| | aminotransferase 2 | 00000175309 | |
| APP | Beta amyloid | Ensembl:ENSG | NP_000475.1 |
| | precursor | 00000142192 | |
| ATP2B1 | Plasma membrane | Ensembl:ENSG | NP_001673.2 |
| | calcium ATPase | 00000070961 | |
| ATP2B4 | Plasma membrane | Ensembl:ENSG | NP_001675.3 |
| | calcium ATPase | 00000058668 | |
| BINI | Bridging | Ensembl:ENSG | NP_647593.1 |
| | integrator | 00000136717 | |
| BPTF/FALZ | Nucleosome | Ensembl:ENSG | NP_872579.2 |
| | remodeling factor | 00000171634 | |
| | subunit | | |
| C12orf41/KANSL2 | Histone acetyl | Ensembl:ENSG | NP_060292.3 |
| | transferase | 00000139620 | |
| | activity | | |
| C14orf133/VIPAR | Sorting of | Ensembl:ENSG | NP_071350.2 |
| | lysosomal | 00000151445 | |
| | proteins | | |
| DMD | Dystrophin | Ensembl:ENSG | NP_003997.1 |
| | | 00000198947 | |
| DTNA | Dystrobrevin | Ensembl:ENSG | NP_001381.2 |
| | | 00000134769 | |
| EIF2C2 | Argonaute | Ensembl:ENSG | NP_036286.2 |
| | translation | 00000123908 | |
| | factor 2C | | |
| EPB41L2 | Transmission | Ensembl:ENSG | NP_001422.1 |
| | across chemical | 00000079819 | |
| | synapses | | |
| FMNL2 | Formin | Ensembl:ENSG | NP_443137.2 |
| | | 00000157827 | |
| GARNL1/RALGAPA1 | Ral GTPase | Ensembl:ENSG | NP_919277.2 |
| | activating | 00000174373 | |
| | protein α | | |
| | subunit | | |
| ITSN2 | Intersectin | Ensembl:ENSG | NP_003015.2 |
| | | 00000198399 | |
| KIAA1217 | Homolog of | Ensembl:ENSG | NP_062536.2 |
| | murine Sickle | 00000120549 | |
| | tail | | |
| LRRFIP1 | Leucine rich | Ensembl:ENSG | NP_001131024.1 |
| | repeat flightless | 00000124831 | |
| | interacting | | |
| MAPT | Microtubule | Ensembl:ENSG | NP_001116538.2 |
| | associated Tau | 00000186868 | |
| MAX | MYC associated | Ensembl:ENSG | NP_002373.3 |
| | factor X | 00000125952 | |
| MEF2A | Myocyte specific | Ensembl:ENSG | NP_005578.2 |
| | enhancer factor | 00000068305 | |
| NCAM1 | Neural cell | Ensembl:ENSG | NP_001229536.1 |
| | adhesion | 00000149294 | |
| | molecule 1 | | |
| PALLD | Palladin | Ensembl:ENSG | NP_001159580.1 |
| | | 00000129116 | |
| PDLIM7 | PDZ and LIM | Ensembl:ENSG | NP_005442.2 |
| | domain | 00000196923 | |
| | Serine/Threonine | | |
| PPP2R5C | Phosphatase 2A | Ensembl:ENSG | NP_001155197.1 |
| | regulatory | 00000078304 | |
| | subunit B56 | | |
| PTPN3 | Tyrosine | Ensembl:ENSG | NP_002820.3 |
| | phosphatase | 00000070159 | |
| | non receptor | | |
| RPGR | Retinitis | Ensembl:ENSG | NP_001030025.1 |
| | pigmentosa | 00000156313 | |
| | GTPase regulator | | |
| RRN3 | RNA | Ensembl:ENSG | NP_060897.3 |
| | polymerase 1 | 00000085721 | |
| | specific | | |
| | transcription | | |
| | factor | | |
| SAD1/BRSK2 | Serine threonine | Ensembl:ENSG | NP_115806.1 |
| | kinase SAD | 00000174672 | |
| SAMD4A | Smaug | Ensembl:ENSG | NP_056404.4 |
| | homolog 1 | 00000020577 | |
| SEMA6D | Semaphorin 6D | Ensembl:ENSG | NP_705871.1 |
| | | 00000137872 | |
| SLC25A3 | Phosphate carrier | Ensembl:ENSG | NP_005879.1 |
| | protein, | 00000075415 | |
| | mitonchondrial | | |
| SLC39A9 | NFR2 pathway, | Ensembl:ENSG | NP_060845.2 |
| | may act as a | 00000029364 | |
| | zinc-influx | | |
| | transporter | | |
| SMTN | Smoothelin | Ensembl:ENSG | NP_001193946.1 |
| | | 00000183963 | |
| SORBS1 | Sorbin and SH3 | Ensembl:ENSG | NP_001030126.1 |
| | domain | 00000095637 | |
| | containing 1 | | |
| STXBP5 | Tomosyn | Ensembl:ENSG | NP_640337.3 |
| | | 00000164506 | |
| SVIL | Archvillin; | Ensembl:ENSG | NP_068506.2 |
| | Supervilin, | 00000197321 | |
| TPM1 | Tropomyosin | Ensembl:ENSG | NP_001018004.1 |
| | | 00000140416 | |
| TPM3 | Tropomyosin | Ensembl:ENSG | NP_689476.2 |
| | | 00000143549 | |
| TRIM66 | Tripartite motif | Ensembl:ENSG | NP_055633.1 |
| | containing 66 | 00000166436 | |
| TTN | Titin | Ensembl:ENSG | NP_001254479.2 |
| | | 00000155657 | |
| VPS29 | Vacuolar protein | Ensembl:ENSG | NP_476528.1 |
| | sorting 29 | 00000111237 | |
| XPNPEP1 | Aminoacyl | Ensembl:ENSG | NP_065116.3 |
| | proline | 00000108039 | |
| | aminopeptidase | | |
| ZMYND8 | Protein kinase C | Ensembl:ENSG | NP_898868.1 |
| | binding | 00000101040 | |
| | protein 1 | | |

* Representative protein in the family encoded by the gene.

The hnRNP L pathway is highly enriched in genes that are linked to autism spectrum disorder (ASD, 12 targets/46). Based on this discovery, isoprenoid antibiotics, including but not limited to the compounds ascochlorin, and its derivatives (i.e. natural and synthetic related compounds, e.g. 4-O-methyl ascochlorin (MAC), the ascochlorin glycoside Vertihemipterin A, and its aglycone, 4′,5′-dihydro-4′-hydroxyascochlorin, 8′-hydroxyascochlorin; LL-Z1272delta, 8′,9′-dehydroascochlorin; ascofuranone; ascofuranol; AS-6; Cylindrol A5) can be used directly, and/or as chemical template structures, to treat autism spectrum disorder and related neurological/psychiatric disorders, including but not limited to, mental retardation, learning disability, attention deficit hyperactivity disorder, dyslexia, epilepsy, bipolar disorder, and schizophrenia. Isoprenoid antibiotics have been shown to increase hnRNP L protein levels 12× in vitro (Kang et al., J Proteome Res. 2006 October; 5(10):2620-3).

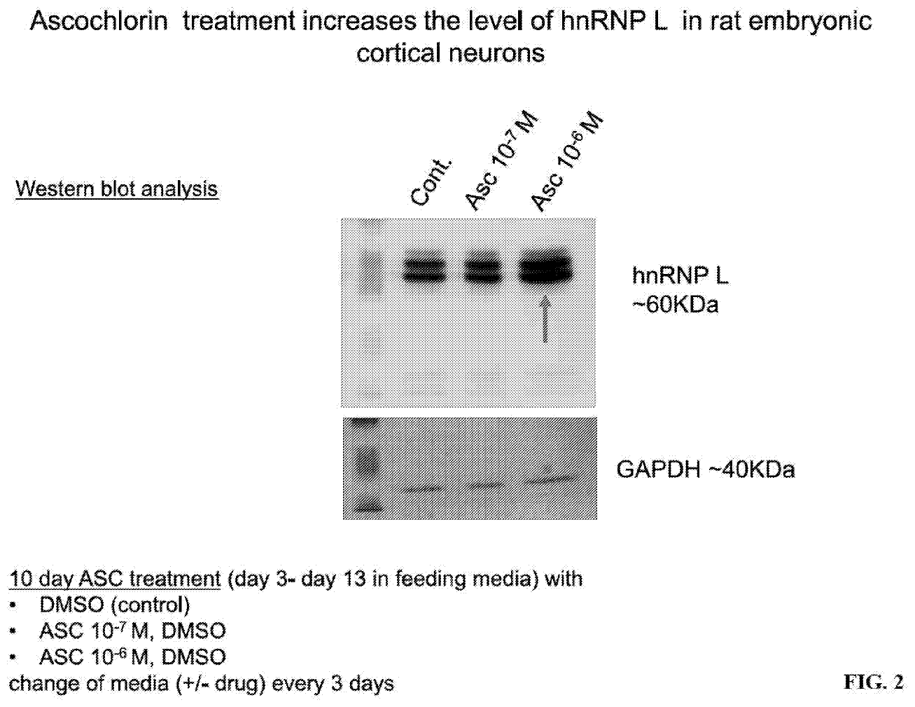

The hnRNP L pathway is useful to identify genes/targets relevant to the treatment of ASD. For example, a cell-based assay is used to identify drugs that modulate hnRNP L levels. Such a cell-based assay utilizes any relevant cell type, e.g., rat embryonic cortical neurons (see FIG. 2), where hnRNP L plays a role in cell physiology/morphology (including but not limited to, neurons, glia, stem cells, pluripotent/multipotent progenitor cells, or undifferentiated cells). Such assays enable the identification of pharmacological compounds that are useful for the development of ASD therapeutics. In addition to being relevant to ASD, the pathway may be used to identify targets/therapeutic compounds for other neurological/psychiatric disorders, including but not limited to, mental retardation, learning disability, attention deficit hyperactivity disorder, dyslexia, epilepsy, bipolar disorder, and schizophrenia.

Thus, the compounds and methods targeting hnRNP L-regulated pathway are useful as treatment formulations and to identify compound and/or targets for the treatment of autism spectrum disorder and other neurological/psychiatric disorders.

### Isoprenoid Antibiotics

Isoprenoid antibiotics, including but not limited to the compounds ascochlorin, and its derivatives (i.e. natural and synthetic related compounds, e.g. ascofuranone, ascofuranol, MAC, AS-6, cylindrol A₅, vertihemipterin A, vertihemipterin A aglycone, 8′-hydroxyascochlorin, 8′,9′-dehydroaschchlorin, 8′-acetoxyascochlorin, colletochlorin) can be used directly, and/or as chemical template structures, to treat autism, autism spectrum disorder and related neurological and psychiatric disorders, including but not limited to, mental retardation, learning disability, attention deficit hyperactivity disorder, dyslexia, epilepsy, bipolar disorder, and schizophrenia.

Ascochlorin been shown to increase hnRNP L protein levels in vitro, in U2OS osteosarcoma cells (Kang et al., J Proteome Res. 2006 October; 5(10):2620-3). Surprisingly, ascochlorin has been shown to increase hnRNP L levels in neurons (e.g., primary rat cortical neurons, FIG. 2), a cell/tissue type significantly different from osteosarcoma tissue. In addition, the hnRNP L pathway can be utilized to identify novel genes/targets relevant to the treatment of autism spectrum disorder. A cell-based assay (e.g., assessing drug-induced changes in hnRNP L levels), optimized in cell types where hnRNP L plays a role in cell physiology/morphology (including but not limited to, neurons, glia, stem cells, pluripotent/multipotent progenitor cells, or undifferentiated cells), enables the identification of pharmacological compounds that are useful for the development of autism spectrum disorder therapeutics. In addition to ASD, this screening strategy is useful to identify novel targets/pre-therapeutic leads for other neurological and psychiatric disorders, including but not limited to, mental retardation, intellectual disability, learning disability, attention deficit hyperactivity disorder, dyslexia, epilepsy, bipolar disorder, and schizophrenia.

### Autism Spectrum Disorder

Autism spectrum disorder (ASD) is a complex neurodevelopmental disorder that is typically recognized in early childhood and has a lifelong course (Lacivita et al., J. Med. Chem. 2017, 60 (22), 9114-9141 and references cited therein). According to the latest diagnostic criteria, it is characterized by two core symptoms: (1) persistent deficits in social communication and social interaction, (2) restricted, repetitive patterns of behavior, interests, and activities. The diagnosis is based on clinical observation and further established by standardized testing of the patient with the Autism Diagnostic Observation Schedule 2, and/or by parental interview with the Autism Diagnostic Interview-Revised. Thus far, no behavioral, neuroimaging, electrophysiological, or genetic tests can specifically diagnose ASD. Comorbid conditions such as intellectual disability, seizures, and sleep problems are frequent, whereas anxiety, depression, and obsessive-compulsive disorder (OCD) are less frequent.

ASD distinguishes from most other behavioral disorders for the impressive clinical and pathogenetic heterogeneity, which has led to the designation with the term ASD of a set of neurodevelopmental disorders with early onset in life, sharing autism as a common feature, but caused by separate processes. Originally, ASD was believed to be relatively rare, but the prevalence rates have dramatically increased in the past decade, from approximately 4/10000 to 1/68 children. Various reasons have been put forward to account for this dramatic increase, including broadening of the spectrum to include even milder forms, improved clinical detection, and higher public awareness. As a result, ASD has recently emerged as a major public health issue worldwide.

Altered neurodevelopment during the first and second trimesters of prenatal life is believed to be an underlying neuropathological cause of ASD. Post-mortem studies have unveiled neuroanatomic and cytoarchitectonic aberrations in various brain regions, including cerebellum, hippocampus, inferior olivary complex, amygdala, entorhinal cortex, fusiform gyrus, and anterior and posterior cingulate cortex, with increased growth of the frontal lobes, thinner cortical minicolumns, and increased dendritic spine density. While some of these aberrations (e.g., reduced programmed cell death and/or increased cell proliferation, altered cell migration, abnormal cell differentiation with reduced neuronal body size, abnormal neurite sprouting, pruning that cause atypical wiring into the brain, reduced synapse formation and delayed myelination) appear to be related to alterations occurring during early pregnancy, corresponding neurodevelopmental processes and accompanying brain plasticity are active well into late prenatal and postnatal life (Bryan Kolb and Robbin Gibb, Brain Plasticity and Behaviour in the Developing Brain, J Can Acad Child Adolesc Psychiatry. 2011 November; 20(4): 265-276). The observed abnormal neuronal wiring was previously thought to be characterized by long-range hypoconnectivity and local hyperconnectivity. Recent studies have instead shown that abnormal neuronal wiring is characterized by a highly individualized combination of hyper- and hypoconnectivity specific to each ASD patient.

The neurocognitive phenotype of ASD is the result of a complex and highly heterogeneous set of genetic and environmental causes. However, in some patients, the disorder is the result of purely genetic causes due to known chromosomal aberrations or mutations, while in other patients, the disorder is more likely related to environmental causes, such as prenatal exposure to chemical pollutants, toxins, viruses, or even drugs. To date, hundreds of risk genes have been identified and not a major causative gene, with either rare variants that are highly penetrant or common variants with small effects. It is therefore not surprising that this genetic heterogeneity is not associated with a characteristic neuropathology for ASD. Finally, neuroinflammation in ASD is receiving attention because of the altered expression of neuroinflammatory markers observed in the amniotic fluid, serum, cerebrospinal fluid, and the brain tissue of ASD patients (Lacivita et al., J. Med. Chem. 2017, 60 (22) 9114-9141 and references cited therein).

The present invention relates to the treatment of neurological disorders using isoprenoid (prenyl-phenol) antibiotics, including but not limited to the compounds ascochlorin, its derivatives and analogs (e.g. ascofuranone, ascofuranol, MAC, AS-6, cylindrol A₅, vertihemipterin A, vertihemipterin A aglycone, 8′-hydroxyascochlorin, 8′,9′-dehydroaschchlorin, 8′-acetoxyascochlorin, colletochlorin) which can be used directly, and/or as chemical template structures, to help treat neurological disorders in humans. The relevant neurological and psychiatric disorders include, but are not limited to, autism, autism spectrum disorder, mental retardation, learning disability, intellectual disability, attention deficit hyperactivity disorder, dyslexia, epilepsy, bipolar disorder, and schizophrenia.

Natural Sources of the Isoprenoid Antibiotic Compounds

Isoprenoid antibiotics were originally isolated from the phytopathogenic fungus Ascochyta viciae. (Sasaki, H. et al. J Antibiot (Tokyo), 1973, 26:676-680). Among them, ascochlorin and ascofuranone have been shown to be non-toxic compounds. Structurally related compounds have been subsequently isolated from other fungi (e.g., Fusarium, Cylindrocladium, Cylindrocladium ilicicola, Nectria coccinea, Colletotrichum nicotianae, Acremonium luzulae, Cephalosporium diospyri, Verticillium, Cylindrocarpon lucidum, Nigrosabulum globosum, and the insect pathogenic fungus Verticillium hemipterigenum). (Hosono, K. et al. J Antibiot (Tokyo), 2009, 62:571-574; Seephonkai, P. et al. J Antibiot (Tokyo), 2004, 57:10-16).

### Physiological Properties of the Isoprenoid Antibiotic Compounds

Ascochlorin and derivatives (e.g., MAC) as well as analogs (e.g., ascofuranone) display antitumorigenic properties, both in vitro and in vivo (Min-Wen et al., Adv Protein Chem Struct Biol. 2017; 108: 199-225).

In addition to anticancer properties, ascochlorin and its derivatives exhibit a wide range of physiological activities, including antimicrobial/antiviral activity, trypanocidal properties, hypolipidemic activity, suppression of hypertension, improvement of type I and II diabetes, anti inflammatory, and immunomodulation. (Yabu, Y. et al. Parasitol Int. 2003, 52:155-164; Hosono, K. et al. J Antibiot (Tokyo), 2009, 62:571-574; Lee et al., J Cell Biochem. 2016 April; 117(4):978-87; Shen et al., Eur J Pharmacol. 2016 Nov. 15; 791:205-212).

Other examples of ascochlorin derivatives may be found in:
(i) Pubchem databases: http://www.ncbi.nlm.nih.gov/pccompound/?term=ascochlorin; by using the “similar compounds” interactive link;
(ii) Pharmaceutical composition and method of using the same. U.S. Pat. No. 3,995,061.
(iii) Ascochlorin derivatives, and pharmaceutical composition containing the same. U.S. Pat. No. 4,500,544;
(iv) Pyridyl carbonyl ascochlorin derivatives and pharmaceutical compositions containing the same. U.S. Pat. No. 4,542,143;
(v) Ligands of nuclear receptor. U.S. Pat. No. 6,605,639.
(vi) Ascochlorin derivative and use thereof as ampk activator. WO 2017/119515.
(vii) R. A. West, et al., African trypanosomiasis: Synthesis & SAR enabling novel drug discovery of ubiquinol mimics for trypanosome alternative oxidase, European Journal of Medicinal Chemistry 141 (2017) 676-689, https://doi.org/10.1016/j.ejmech.2017.09.067.
(viii) Togashi, M et al., Ascochlorin Derivatives as Ligands for Nuclear Hormone Receptors. J. Med. Chem. 2003; 46:4113-4123.
(ix) Saimoto, H et al., Pharmacophore Identification of Ascofuranone, Potent Inhibitor of Cyanide-Insensitive Alternative Oxidase of Trypanosoma brucei. J. Biochem. 2013; 153:267-273.
   Additional compounds include an ascochlorin derivative from Cylindrocarpon sp. FKI-4602. Kawaguchi et al., J Antibiot (Tokyo). 2013 January; 66(1):23-9; ascochlorin derivatives from the leafhopper pathogenic fungus Microcera sp. BCC 17074. Isaka et al., J Antibiot (Tokyo). 2015 January; 68(1):47-51; and compound described in Discovery of a new structural class of competitive hDHODH inhibitors with in vitro and in vivo anti-inflammatory, immunosuppressive effects. Shen et al., Eur J Pharmacol. 2016 Nov. 15; 791:205-212.

Splicing Factor hnRNP L and Downstream RNA Targets:

hnRNP L binds to RNA targets, for example, the following sequence:

Proteome analysis has demonstrated that ascochlorin treatment of human osteosarcoma cells (U20S) results in a ≥10 fold increase in the levels of three proteins, including the splicing factor hnRNP L (first most upregulated protein, 12×), as well as BIN1 (third most upregulated protein, 10×) (Kang J. H. et al. J Proteome Res. 2006; 5:2620-2631). It has been determined by bioinformatics analysis that BIN1 is a candidate target of hnRNP L. In addition, the BIN1 gene was shown to be associated with autism spectrum disorder (Connolly J. J. et al. Child Dev. 2013 January-February; 84(1):17-33).

The methylated derivative of ascochlorin, 4-O-methylascochlorin (MAC), increases the expression of vascular endothelial growth factor (VEGF) and glucose transporter 1 (GLUT-1) (Jeong J. H. et al. Biochem Biophys Res Commun. 2011; 406:353-358). Both VEGF and GLUT-1 RNAs are well-established targets of hnRNP L (Hamilton B. J. et al. Biochem Biophys Res Commun. 1999; 261:646-651; Ray P. S. et al. Nature. 2009; 457:915-919; Shih S. C. et al. J Biol Chem. 1999; 274:1359-1365).

Ascochlorin and/or its derivatives promote the maintenance of normal brain physiology by targeting hnRNP L and/or components of the coordinated hnRNP L-regulated pathway(s). The compounds and methods of the invention provide therapeutic interventions to reduce the symptoms of autism spectrum disorder and additional neurological and psychiatric disorders by addressing underlying causes, e.g., aberrant splicing defects in the genes identified herein.

### RBFox1

The RNA binding protein RBFox1 binds to the hexamer UGCAUG (Lee, et al., Neuron 89(1):113-28, 2016; Auweter, et al. EMBO J. 25(1):163-73, 2006). Mutations in the conserved splicing factor FOX1 are linked to autism spectrum disorder (Davis L. K. et al. Am J Med Genet A. 2012; 158A:1654-1661; Martin C. L. et al. Am J Med Genet B Neuropsychiatr Genet. 2007; 144B:869-876; Voineagu I. et al. Nature. 2011; 474:380-384).

### Identification of Compounds to Treat Autism Spectrum Disorder

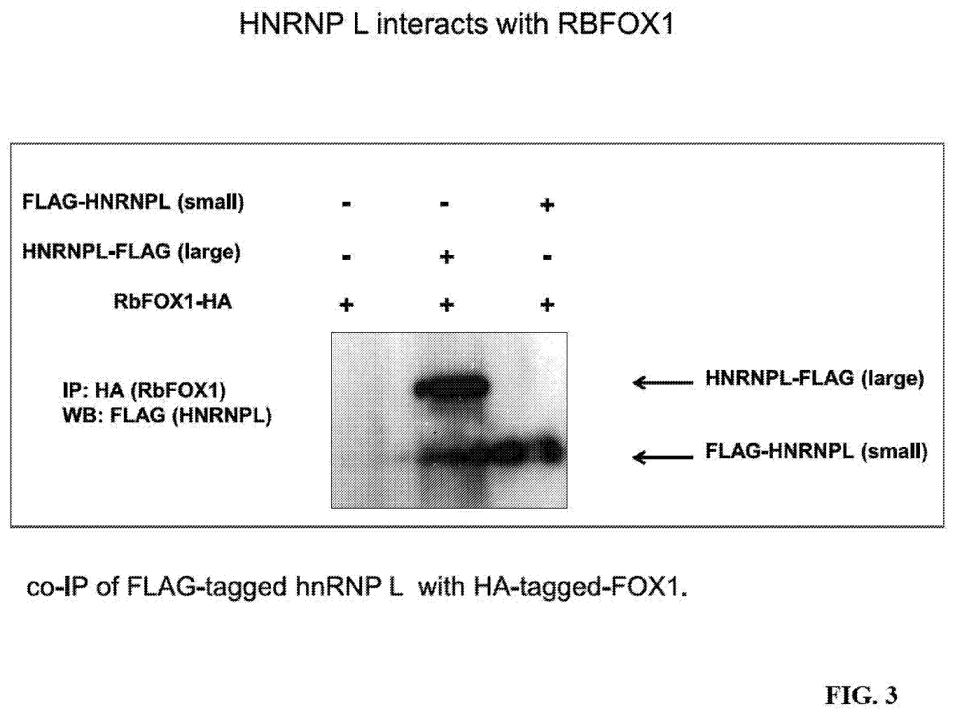

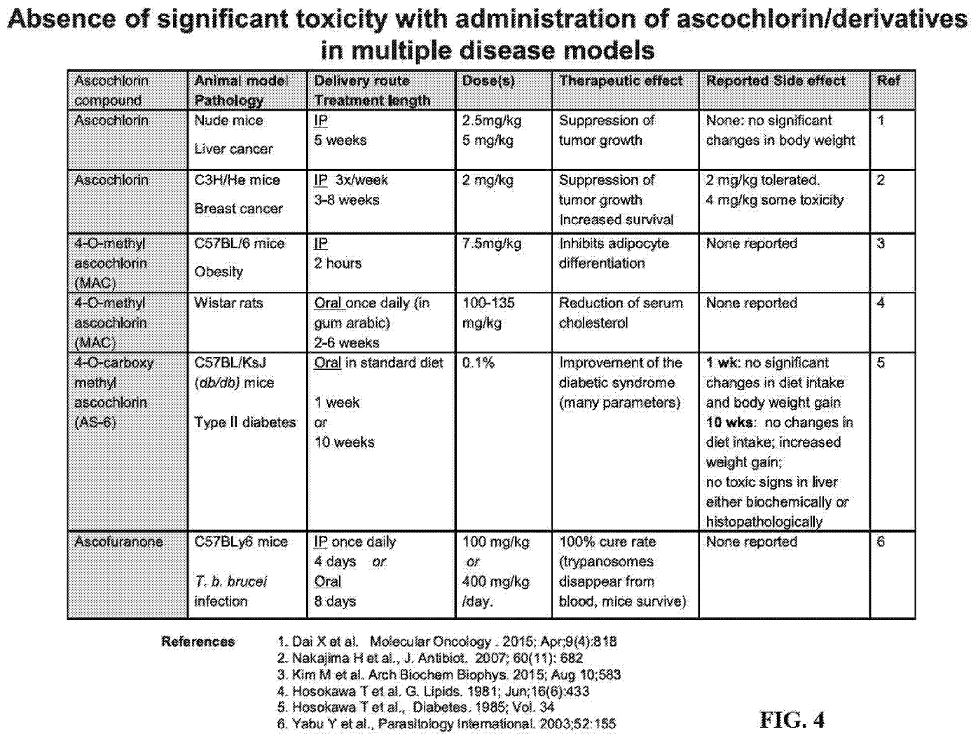

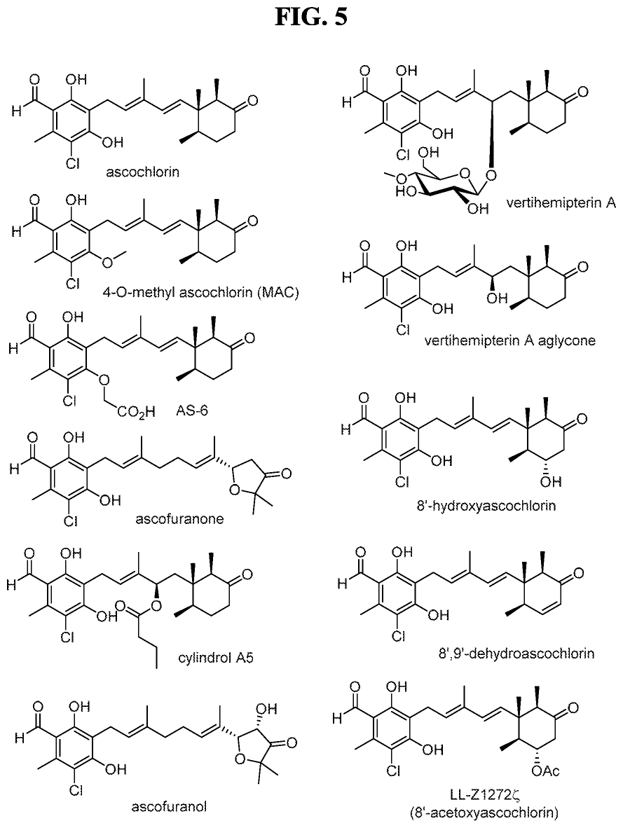

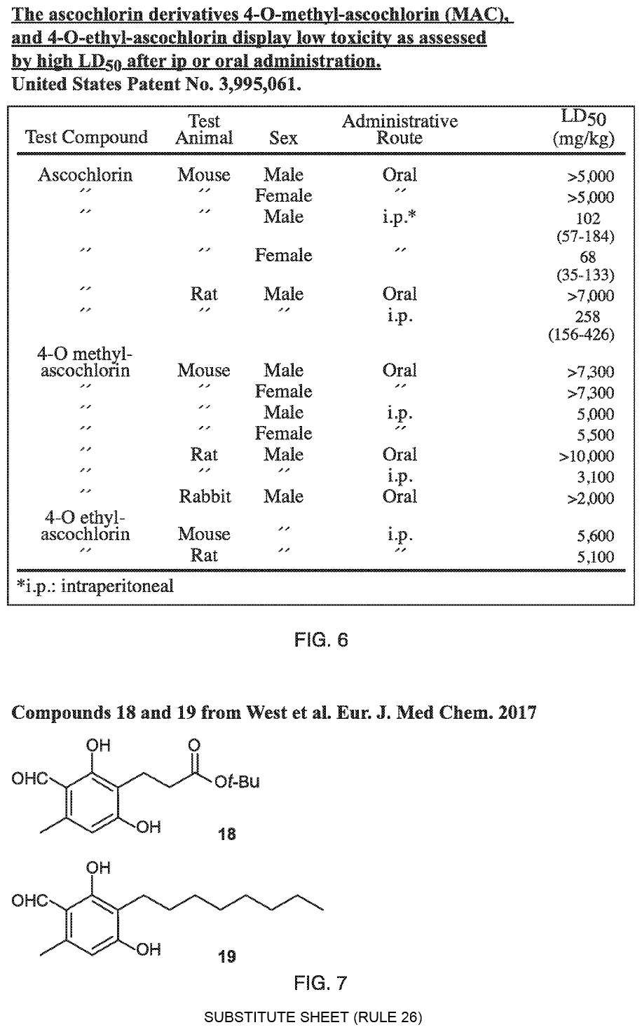

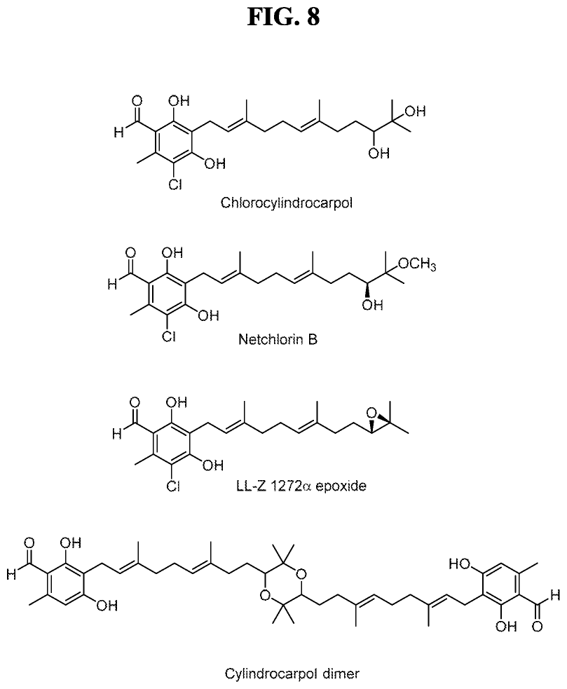

Compounds that modify hnRNP L levels therefore hold promise as lead structures for the development of ASD/mental retardation/neurological disorders. Using Western Blot analysis, it has been observed that ascochlorin treatment of cultured rat primary cortical neurons results in increased levels of hnRNP L. FIG. 2 is a photograph of an electrophoretic gel showing how ascochlorin treatment increases the level of hnRNP L in rat embryonic cortical neurons (hnRNP L 60 KDa). FIG. 2 also shows that ascochlorin treatment does not affect the level of the control protein in rat embryonic cortical neurons (GADPH 40 KDa). FIG. 3 indicates that HNRNP L interacts with RBFOX1.

Ascochlorin and/or its derivatives can promote the maintenance of normal brain physiology by targeting hnRNP L and/or components of the coordinated hnRNP L-regulated pathway(s). The compounds and methods described herein provide pharmacological leads to help treat autism spectrum disorder and additional neurological and psychiatric disorders. General Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the term “about” in the context of a numerical value or range means ±10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

A small molecule is a compound that is less than 2000 daltons in mass. The molecular mass of the small molecule is preferably less than 1000 daltons, more preferably less than 600 daltons, e.g., the compound is less than 500 daltons, 400 daltons, 300 daltons, 200 daltons, or 100 daltons.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. A purified protein or polypeptide is free of the amino acids that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “subject,” “patient,” “individual,” and the like as used herein are not intended to be limiting and can be generally interchanged. That is, an individual described as a “patient” does not necessarily have a given disease, but may be merely seeking medical advice.

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

As used herein, “treating” encompasses, e.g., inhibition, regression, or stasis of the progression of a disorder. Treating also encompasses the prevention or amelioration of any symptom or symptoms of the disorder. As used herein, “inhibition” of disease progression or a disease complication in a subject means preventing or reducing the disease progression and/or disease complication in the subject.

As used herein, a “symptom” associated with a disorder includes any clinical or laboratory manifestation associated with the disorder, and is not limited to what the subject can feel or observe.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

As used herein, “pharmaceutically acceptable” carrier or excipient refers to a carrier or excipient that is suitable for use with humans and/or animals without undue adverse side effects (such as toxicity, irritation, and allergic response) commensurate with a reasonable benefit/risk ratio. It can be, e.g., a pharmaceutically acceptable solvent, suspending agent or vehicle, for delivering the instant compounds to the subject.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

The term “neurological disorder or disease” as used herein refers to a disorder, disease or condition which directly or indirectly affects the normal functioning or anatomy of a subject's nervous system, including, but not limited to, the brain. In one embodiment, the neurological disorder or disease is a neurodevelopmental disorder.

An example of a neurological disorder or disease is autism. Another example of a neurological disorder or disease is autism spectrum disorder. In other examples, the neurological disorder or disease is epilepsy, schizophrenia or mental retardation.

Autism spectrum disorder (ASD) is a range of complex neurodevelopment disorders, characterized by social impairments, communication difficulties, and restricted, repetitive, and stereotyped patterns of behavior. Autism (also known as autistic disorder or classical ASD) is the most severe form of ASD. Other conditions along the spectrum include Asperger syndrome, childhood disintegrative disorder and pervasive developmental disorder not otherwise specified (also referred to as PDD-NOS), and Chromosome 15q11.2-13.1 duplication syndrome (dup15q syndrome).

The phrase “treating a neurological disorder or disease” as used herein includes, but is not limited to, reversing, alleviating or inhibiting the progression of a neurological disorder or disease or conditions associated with a neurological disorder or disease. As used herein, and as well understood in the art, “to treat” or “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

In one embodiment, treating a neurological disorder or disease includes preventing the occurrence of a neurological disorder or disease or symptoms or conditions associated with a neurological disorder or disease or preventing worsening of the severity of a neurological disorder or disease or conditions associated with a neurological disorder or disease.

The term “neurological function” as used herein refers to the functioning and/or activity of a subject's nervous system.

The term “improving neurological function” as used herein refers to improving the structure, function and/or activity of a subject's nervous system. In one embodiment, improving neurological function includes improving neurodevelopment and/or improving behavior.

The term “subject” as used herein refers to any member of the animal kingdom, such as a mammal. In one embodiment, the subject is a human. In another embodiment, the subject is a mouse.

The term “a cell” includes a single cell as well as a plurality or population of cells. Administering a modulator or an agent to a cell includes both in vitro and in vivo administrations.

The modulators and agents described herein may be formulated into pharmaceutical compositions for administration to subjects and/or use in subjects in a biologically compatible form suitable for administration in vivo. The compositions described herein can be prepared by per se known methods for the preparation of pharmaceutically acceptable compositions that can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, 20th ed., Mack Publishing Company, Easton, Pa., USA, 2000). On this basis, the compositions include, albeit not exclusively, solutions of the substances in association with one or more pharmaceutically acceptable vehicles or diluents, and contained in buffered solutions with a suitable pH and iso-osmotic with the physiological fluids.

In one embodiment, the modulators and agents described herein are formulated into pharmaceutical compositions for administration to the brain or central nervous system of a subject. Modulators, agents and pharmaceutical compositions which cannot penetrate the blood-brain barrier can be effectively administered by an intraventricular route or other appropriate delivery system suitable for administration to the brain.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. Proteins may be supplied, for example but not by way of limitation, as a lyophilized powder which is reconstituted with sterile water or saline prior to administration to the patient.

Pharmaceutical compositions may comprise a pharmaceutically acceptable carrier. Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1 (2,3-dioleyloxy)propyl)N,N,N-trimethylammonium chloride (DOTMA), diolesylphosphotidyl-ethanolamine (DOPE), and liposomes. Such compositions should contain a therapeutically effective amount of the compound, together with a suitable amount of carrier so as to provide the form for direct administration to the patient.

The compositions may be in the form of a pharmaceutically acceptable salt which includes, without limitation, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylarnino ethanol, histidine, procaine, etc.

The modulators, agents and/or pharmaceutical compositions described herein may be administered to, or used in, living organisms including humans, and animals. The term “subject” or “animal” as used herein refers to any member of the animal kingdom, in one embodiment a mammal such as a human being.

Administration of an “effective amount” of the modulators, agents and/or pharmaceutical compositions is defined as an amount effective, at dosages and for periods of time necessary to achieve the desired result. For example, an effective amount of a substance may vary according to factors such as the disease state, age, sex, and weight of the individual, and the ability of the recombinant protein to elicit a desired response in the individual. Dosage regime may be adjusted to provide the optimum therapeutic response. For example, several divided doses may be administered daily or the dose may be proportionally reduced as indicated by the exigencies of the therapeutic situation.

### Treatment of ASD and FOX1

The utility of ascochlorin (which increases hnRNP L levels ˜12× (Kang J. H. et al. J Proteome Res. 2006; 5:2620-2631) and its derivatives as for the treatment of autism spectrum disorder is underscored by the discovery that hnRNP L directly interacts with FOX1. Pharmacological stabilization of the hnRNP L-FOX1 complex may be beneficial in cases where a decrease in the levels of FOX1 (˜5.9×) is known to cause autism (Voineagu I. et al. Nature. 2011; 474:380-384). Further highlighting the potential of ascochlorin and its derivatives for the treatment neurological disorders is evidence that some antibiotics have ancillary neuroprotective effects (Stock M. L. et al. Neuropharmacology. 2013; 73C:174-182).

In addition to ascochlorin, the compounds and compositions described below are useful for the treatment of ASD.

Compounds of Formula 1
Formula 1

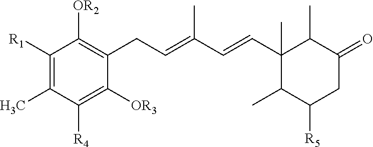

| Compound # | R₁ | R₂ | R₃ | R₄ | R₅ |
|---|---|---|---|---|---|
| 1 | CHO | H | H | Cl | H |
| 2 | CHO | H | H | Cl | OAc |
| 3 | CHO | H | H | Br | H |
| 4 | CHO | H | H | H | H |
| 5 | CHO | H | CH₃CO | Cl | H |
| 6 | CHO | H | CH₃ | Cl | H |
| 7 | CHO | CH₃ | CH₃ | Cl | H |
| 8 | CHO | CH₃CO | CH₃ | Cl | H |
| 9 | CHO | CH₃ | CH₃CO | Cl | H |
| 10 | CHO | CH₃ | H | Cl | H |
| 11 | CHO | H | CH₃CH₂ | Cl | H |
| 12 | CHO | H | Allyl | Cl | H |
| 13 | CHO | H | Butyl | Cl | H |
| 14 | CHO | H | CH₂COOH | Cl | H |
| 15 | CHO | H | (CH₂)₂COOH | Cl | H |
| 16 | CHO | H | (CH₂)₃COOH | Cl | H |
| 17 | CHO | H | (CH₂)₄COOH | Cl | H |
| 18 | CHO | H | CH₂COOCH₃ | Cl | H |
| 19 | CHO | H | Nicotinoyl | Cl | H |
| 20 | CHO | H | Benzoyl | Cl | H |
| 21 | CHO | H | Isonicotinoyl | Cl | H |
| 22 | CHO | H | CH₂COOC₂H₅ | Cl | H |
| 23 | CHO | H | CH₂COOCH₃ | Cl | H |
| 24 | CHO | H | CH₂COOH | Cl | H |
| 25 | CHO | H | CHCH₃COOC₂H₅ | Cl | H |
| 26 | CHO | H | CHCH₃COOC₄H₉ | Cl | H |
| 27 | CHO | H | CHCH₂CH₃COOC₂H₅ | Cl | H |
| 28 | CHO | H | (CH₂)₃COOC₂H₅ | Cl | H |
| 29 | CHO | H | CHCH₃COOH | Cl | H |
| 30 | CHO | H | (CH₂)₃COOH | Cl | H |
| 31 | CHO | H | Nicotinoyl | Cl | H |
| 32 | CHO | H | COC₆H₄OCH₃ | Cl | H |
| 33 | CHO | H | COC₆H₄COOCH₃ | Cl | H |
| 34 | CHO | H | CON(C₂H₅) | Cl | H |
| 35 | CHO | H | COCH₂OC₆H₄Cl | Cl | H |
| 36 | CHO | H | Isonicotinoyl | Cl | H |
| 37 | CHO | H | Picolinoyl | Cl | H |
| 38 | CHO | H | CH₃ | Cl | H |
| 39 | C₂H₂COCH₃ | H | H | Cl | O |
| 40 | CHO | H | CH₃CO | H | H |
| 41 | CHO | H | CH₃ | H | H |
| 42 | C₂H₂COCH₃ | H | CH₂COOH | Cl | O |
| 43 | CHO | CH₃CO | CH₃CO | Cl | H |
| 44 | C(OCH₃)₂ | H | CH₃CO | Cl | H |
| 45 | C(OCH₂CH₃)₂ | H | CH₃CO | Cl | H |
| 46 | C(OCH₂CH₃)₂ | H | CH₃ | Cl | H |
| 47 | C(O(CH₂)₃CH₃)₂ | H | CH₃ | Cl | H |
| 48 | 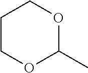 | H | CH₃ | Cl | H |
| 49 | CHO | H | (CH₂)₃CH₃ | Cl | H |
| 50 | CHO | H | CH₂CH₃ | Cl | H |
| 51 | CHO | H | CH₂CHCH₂ | Cl | H |
| 52 | CO₂H | H | H | Cl | H |

Compounds of Formula 2
Formula 2

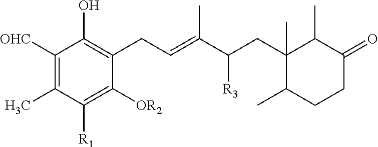

| Compound # | R₁ | R₂ | R₃ |
|---|---|---|---|
| 53 | Cl | H | H |
| 54 | H | H | H |
| 55 | Cl | H | OH |
| 56 | Cl | H | OAc |
| 57 | Cl | CH₃ | H |
| 58 | Cl | CH₃CO | H |
| 59 | H | CH₃ | H |
| 60 | H | CH₃CO | H |
| 61 | Cl | H | OCO(CH₃)₂ |
| 62 | Cl | H | OCOCH₂C(CH₃)₂ |

Additional compounds include, but are not limited to:
3-chloro-4,6-dihydroxy-2-methyl-5-((2E,4E)-3-methyl-5-((1R,2R,6R)-1,2,6-trimethyl-3-oxocyclohexyl)penta-2,4-dien-1-yl)benzaldehyde;
3-chloro-6-hydroxy-4-methoxy-2-methyl-5-((2E,4E)-3-methyl-5-((1R,2R,6R)-1,2,6-trimethyl-3-oxocyclohexyl)penta-2,4-dien-1-yl)benzaldehyde;
2-(2-chloro-4-formyl-5-hydroxy-3-methyl-6-((2E,4E)-3-methyl-5-((1R,2R,6R)-1,2,6-trimethyl-3-oxocyclohexyl)penta-2,4-dien-1-yl)phenoxy)acetic acid;
3-chloro-5-((2E,6E)-7-((S)-5,5-dimethyl-4-oxotetrahydrofuran-2-yl)-3-methylocta-2,6-dien-1-yl)-4,6-dihydroxy-2-methylbenzaldehyde;
(R,E)-5-(3-chloro-5-formyl-2,6-dihydroxy-4-methylphenyl)-3-methyl-1-((1S,2R,6R)-1,2,6-trimethyl-3-oxocyclohexyl)pent-3-en-2-yl butyrate;
3-chloro-4,6-dihydroxy-5-((2E,6E)-7-((2R,3S)-3-hydroxy-5,5-dimethyl-4-oxotetrahydrofuran-2-yl)-3-methylocta-2,6-dien-1-yl)-2-methylbenzaldehyde;
3-chloro-5-((R,E)-4-(((2R,3R,4R,5S,6R)-3,4-dihydroxy-6-(hydroxymethyl)-5-methoxytetrahydro-2H-pyran-2-yl)oxy)-3-methyl-5-((1S,2R,6R)-1,2,6-trimethyl-3-oxocyclohexyl)pent-2-en-1-yl)-4,6-dihydroxy-2-methylbenzaldehyde;
3-chloro-4,6-dihydroxy-5-((R,E)-4-hydroxy-3-methyl-5-((1S,2R,6R)-1,2,6-trimethyl-3-oxocyclohexyl)pent-2-en-1-yl)-2-methylbenzaldehyde;
3-chloro-4,6-dihydroxy-5-((2E,4E)-5-((1S,2S,3S,6R)-3-hydroxy-1,2,6-trimethyl-5-oxocyclohexyl)-3-methylpenta-2,4-dien-1-yl)-2-methylbenzaldehyde;
3-chloro-4,6-dihydroxy-2-methyl-5-((2E,4E)-3-methyl-5-((1S,2R,6R)-1,2,6-trimethyl-5-oxocyclohex-3-en-1-yl)penta-2,4-dien-1-yl)benzaldehyde;
3-chloro-4,6-dihydroxy-5-((2E,4E)-5-((1S,2S,3S,6R)-3-hydroxy-1,2,6-trimethyl-5-oxocyclohexyl)-3-methylpenta-2,4-dien-1-yl)-2-methylbenzaldehyde;
(E)-3-chloro-5-(3,7-dimethylocta-2,6-dien-1-yl)-4,6-dihydroxy-2-methylbenzaldehyde;

cefacetrile; cefotaxime; ciproflaxin; netilimicine; or a quinolone/fluoroquinolone compound.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method for treating or reducing a symptom of Autism Spectrum Disorder (ASD), comprising administering to a subject a composition that either upregulates or downregulates heterogeneous nuclear ribonucleoprotein L (hnRNP L) or a target of hnRNP L selected from the group consisting of BIN1, ABl1, ACSS2, AGAP3, AGXT2L2, APP, ATP2B1, ATP2B4, BPTF, C12orf41, C14orf133, DMD, DTNA, ElF2C2, EPB41L2, FMNL2, GARNL1, ITSN2, KIAA1217, LRRFIP1, MAPT, MAX, MEF2A, NCAM1, PALLD, PDLIM7, PPP2R5C, PTPN3, RPGR, RRN3, SAD1, SAMD4A, SEMA6D, SLC25A3, SLC39A9, SMTN, SORBS1, STXBP5, SVIL, TPM1, TPM3, TRIM66, TTN, VPS29, XPNPEP1, and ZMYND8.

2. The method of claim 1, wherein said target of hnRNP L comprises BIN1.

3. The method of claim 1, wherein said target of hnRNP L further comprises a RBFOX1.

4. The method of claim 1, wherein said composition modulates the level of hnRNP L in a neuronal cell of said subject.

5. The method of claim 1, wherein said composition comprises an isoprenoid antibiotic.

6. The method of claim 1, wherein said composition comprises ascochlorin or a derivative or analog thereof.

7. A method of identifying a compound for treatment of ASD, comprising contacting a neuronal cell with a candidate compound and detecting a change in hnRNP L expression or activity or a change in expression or activity of a downstream target of hnRNP L, wherein an increase or decrease in said expression or activity indicates that said compound decreases a symptom or severity of ASD.

8. The method of claim 7, wherein said a change in hnRNP L expression or activity or a change in expression or activity of a downstream target of hnRNP L comprises an increase in said expression or activity.

9. The method of claim 7, wherein said a change in hnRNP L expression or activity or a change in expression or activity of a downstream target of hnRNP L comprises a decrease in said expression or activity.

10. A method for identifying a subject suffering from or at risk of developing ASD, comprising:
i) detecting a nucleic acid mutation in hnRNP L in a tissue of said subject; or
ii) detecting a decrease in hnRNP L level in a tissue of said subject compared to a normal control hnRNP L level, wherein a change of at least 10% compared to said normal control level indicates that said subject comprises or is at risk of developing ASD.

11. (canceled)

12. The method of claim 9, further comprising administering to said subject a composition that alters expression of heterogeneous nuclear ribonucleoprotein L (hnRNP L) or a target of hnRNP L selected from the group consisting of ABl1, ACSS2, AGAP3, AGXT2L2, APP, ATP2B1, ATP2B4, BIN1, BPTF, C12orf41, C14orf133, DMD, DTNA, ElF2C2, EPB41L2, FMNL2, GARNL1, ITSN2, KIAA1217, LRRFIP1, MAPT, MAX, MEF2A, NCAM1, PALLD, PDLIM7, PPP2R5C, PTPN3, RPGR, RRN3, SAD1, SAMD4A, SEMA6D, SLC25A3, SLC39A9, SMTN, SORBS1, STXBP5, SVIL, TPM1, TPM3, TRIM66, TTN, VPS29, XPNPEP1, and ZMYND8.

13. The method of claim 12, wherein said target of hnRNP L further comprises RBFOX1.

14. A composition comprising an isoprenoid antibiotic for use in the method of claim 1.

15. The composition of claim 14, wherein said antibiotic comprises ascochlorin or a derivative thereof.

16. The method of claim 1, wherein hnRNP L interacts with RBFOX1.

17. The method of claim 12, wherein hnRNP L interacts with RBFOX1.


SEQUENCE LISTING (13 sequences)

SEQ ID NO: 1 - DNA, 20 bp, Artificial Sequence
  Description of Artificial Sequence Synthetic oligonucleotide
atacatgacacacacacgca

SEQ ID NO: 2 - DNA, 19 bp, Artificial Sequence
  Description of Artificial Sequence Synthetic oligonucleotide
cacacacactacaccacac

SEQ ID NO: 3 - DNA, 20 bp, Artificial Sequence
  Description of Artificial Sequence Synthetic oligonucleotide
cacaccacaacggcaccaca

SEQ ID NO: 4 - DNA, 20 bp, Artificial Sequence
  Description of Artificial Sequence Synthetic oligonucleotide
tgcatctacgtacacacaca

SEQ ID NO: 5 - DNA, 20 bp, Artificial Sequence
  Description of Artificial Sequence Synthetic oligonucleotide
aatcacatacgctttacaca

SEQ ID NO: 6 - DNA, 20 bp, Artificial Sequence
  Description of Artificial Sequence Synthetic oligonucleotide
ccggacaaacacacgaacac

SEQ ID NO: 7 - DNA, 20 bp, Artificial Sequence
  Description of Artificial Sequence Synthetic oligonucleotide
tgcaaatgaaatgactacac

SEQ ID NO: 8 - DNA, 20 bp, Artificial Sequence
  Description of Artificial Sequence Synthetic oligonucleotide
ttcacctttaccggcagcct

SEQ ID NO: 9 - DNA, 21 bp, Artificial Sequence
  Description of Artificial Sequence Synthetic oligonucleotide
tgcaaacctaaatgaggtaca

SEQ ID NO: 10 - DNA, 20 bp, Artificial Sequence
  Description of Artificial Sequence Synthetic oligonucleotide
aaaccaccccaaacaacaac

SEQ ID NO: 11 - DNA, 20 bp, Artificial Sequence
  Description of Artificial Sequence Synthetic oligonucleotide
tttacgactctgcacgttaa

SEQ ID NO: 12 - DNA, 20 bp, Artificial Sequence
  Description of Artificial Sequence Synthetic oligonucleotide
atgcagccctcgtcattggc

SEQ ID NO: 13 - DNA, 20 bp, Artificial Sequence
  Description of Artificial Sequence Synthetic oligonucleotide
ttgttccgatatagcgcgac